(19) United States
(12) Patent Application Publication
(10) Pub. No.: US 2026/0364099 A1
(43) Pub. Date: Nov. 19, 2026

(54) GLYCYRRHIZIC ACID HYDROGELS

(71) Applicants: KATHOLIEKE UNIVERSITEIT LEUVEN, LEUVEN (BE); PURNA PHARMACEUTICALS NV, PUURS (BE)

(72) Inventors: Maarten MEES, Horebeke (BE); Hans STEENACKERS, Leuven (BE); Karin THEVISSEN, Bierbeek (BE); Wim THIELEMANS, Kortrijk (BE); Thijs VACKIER, Dendermonde (BE)

(21) Appl. No.: 19/475,261

(22) PCT Filed: Apr. 15, 2024
(86) PCT No.: PCT/EP2024/060192

§ 371 Date: Oct. 14, 2025

(30) Foreign Application Priority Data
Apr. 14, 2023 (EP) 23168100.8

(51) Int. Cl. A61K 31/704; A61K 9/06; A61P 31/04

(52) U.S. Cl. CPC A61K 31/704; A61K 9/06; A61P 31/04

(57) ABSTRACT

The present application relates to a glycyrrhizic acid hydrogel with a pH of between 2.5 and 5.0, for use in the treatment or prevention of a bacterial infection. The application further relates to a pharmaceutical composition of said hydrogel and a method of treating or preventing a bacterial infection in an individual comprising the step of administering an effective amount of glycyrrhizic acid hydrogel, a pharmaceutically acceptable salt or solvate thereof, with a pH of between 2.5 and 5.0.

### FIELD OF THE INVENTION

The present invention relates to hydrogel compositions comprising glycyrrhizic acid and analogues thereof, within a specific acidic pH range, and the use of such compositions in the treatment or prevention of skin and soft tissue infections and for use as a coating to prevent infections on medical devices.

### BACKGROUND OF THE INVENTION

Different therapeutic properties of glycyrrhizic acid have been reported: anti-viral, anti-inflammatory, antitumoral and hepatoprotective [Azaz & Segal (1980) Pharm Acta Helv. 55, 183-186]. Due to the amphiphilic nature of glycyrrhizic acid, aggregates are formed in aqueous solution eventually leading to gel formation. The formation of a hydrogel of glycyrrhizic acid is related to its acidity and the concentration of glycyrrhizic acid.

Different studies are available on the antibacterial potential of glycyrrhizic acid in solution—not as a hydrogel—with reported activities against: Staphylococci, Enterococci, Helicobacter pylori, Bacillus subtitilis and Pseudomonas aeruginosa. In contrast, several studies also reported the lack of activity of glycyrrhizic acid in solution against Escherichia coli, Proteus vulgaris, Candida albicans and Streptococci. Zhao et al. (2020) ACS Appl Bio Mater 3, 648-653] evaluated the antibacterial activity of a 0.13 wt % glycyrrhizic acid hydrogel in PBS with reported activity against Staphylococcus aureus but with no effect against Escherichia coli.

US 2021/0015965 discloses glycyrrhizic acid hydrogels for wound healing.

### SUMMARY OF THE INVENTION

The present invention discloses methods of increasing the antibacterial/bactericidal activity and the activity spectrum of glycyrrhizic acid hydrogels, by decreasing the pH of the gel below pH 5.0, more specifically between pH 3.0 and pH 4.85.

The resulting antibacterial/bactericidal activity of the glycyrrhizic acid hydrogels cannot be mimicked by reducing the pH of the growth medium or a comparable concentration of glycyrrhizic acid in solution, that is to say not a hydrogel. Hence, it is the combination of the glycyrrhizic acid hydrogel (and not glycyrrhizic acid in solution) at a certain pH that results in the unexpected broad antibacterial/bactericidal activity of these hydrogels.

The present invention demonstrates that decreasing the pH of the hydrogel increases the antibacterial activity against bacterial strains against which glycyrrhizic acid in solution (at pH 7) is not active. Decreasing the pH of the glycyrrhizic acid hydrogel to pH 4.2 already increases the antibacterial activity against Acinetobacter baumannii, Methicillin-resistant Staphylococcus aureus (MRSA), Staphylococcus epidermidis, Streptococcus pyogenes and Enterococcus faecalis. Further decreasing the pH of the glycyrrhizic acid hydrogel to pH 3.8 broadens the activity spectrum to include Pseudomonas aeruginosa. Finally, decreasing the pH of the glycyrrhizic acid hydrogel even further to pH 3 broadens the activity spectrum of the hydrogel to include Escherichia coli, Klebsiella aerogenes and Klebsiella pneumoniae.

### DETAILED DESCRIPTION

### Figure Legends

FIG. 1 Decreasing the pH of the GLY-hydrogel increases the activity spectrum.

FIG. 2 Growth inhibiting activity of Hydrogel A

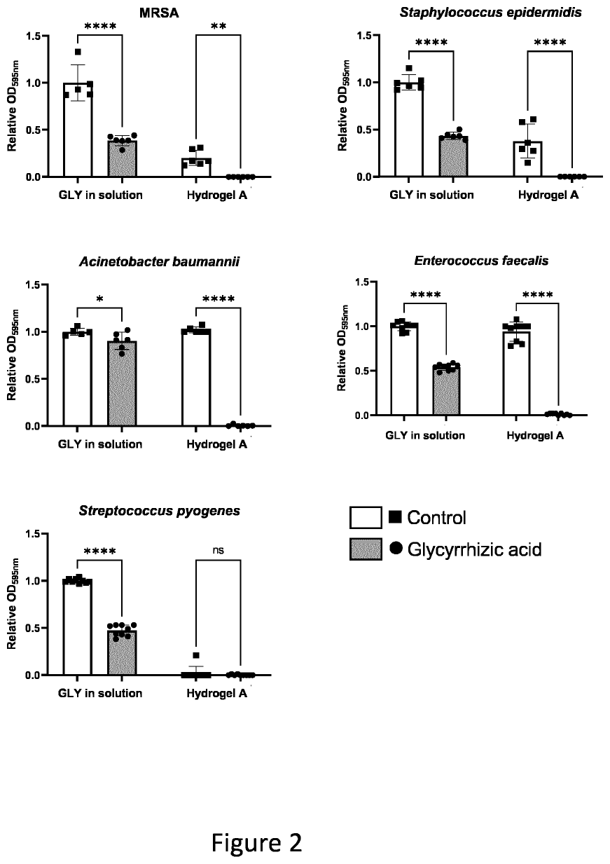

FIG. 3 Bactericidal activity of Hydrogel A

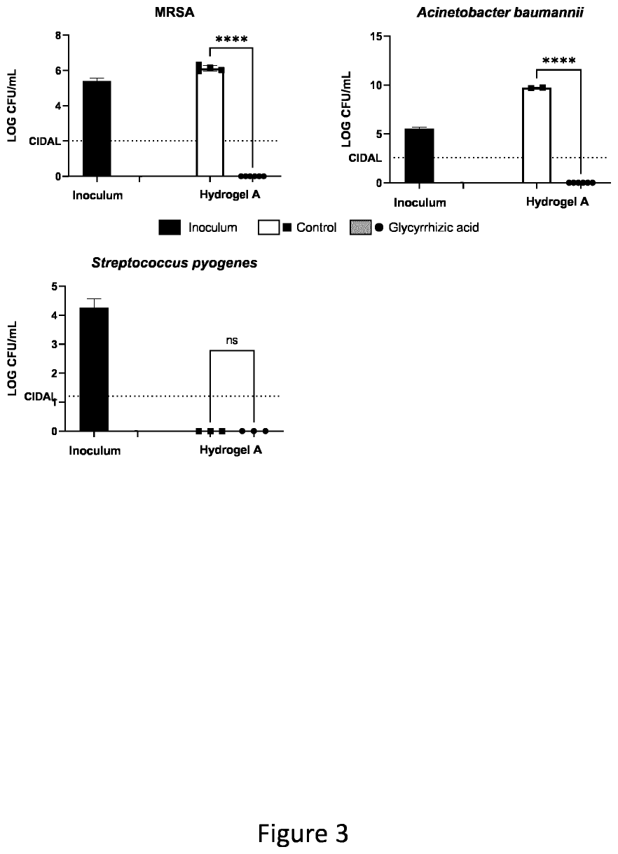

FIG. 4 Growth inhibiting activity of Hydrogel B

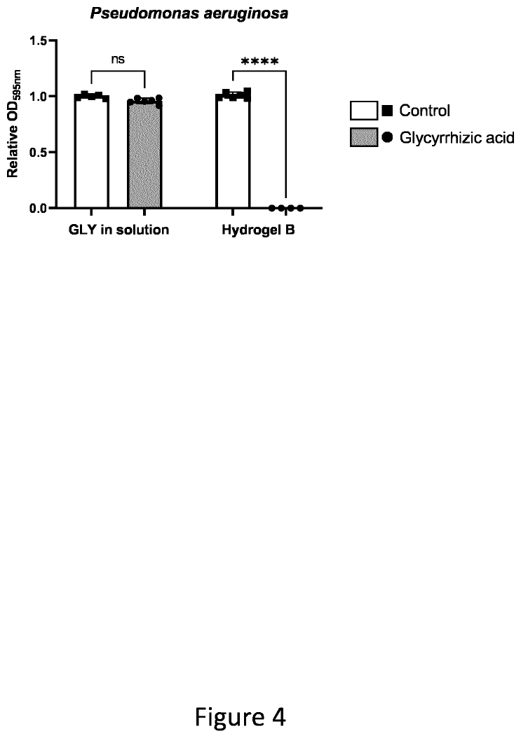

FIG. 5 Bactericidal activity of Hydrogel B

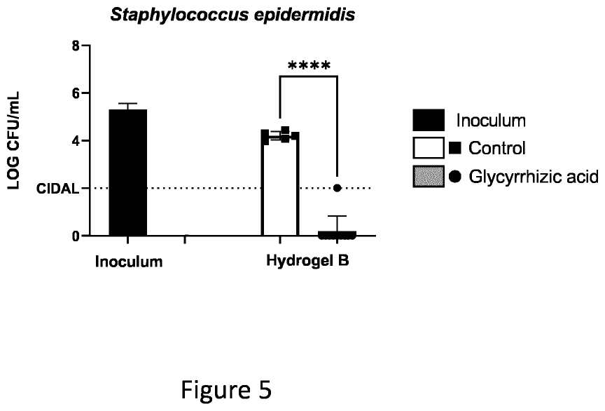

FIG. 6 Growth inhibiting activity of Hydrogel C

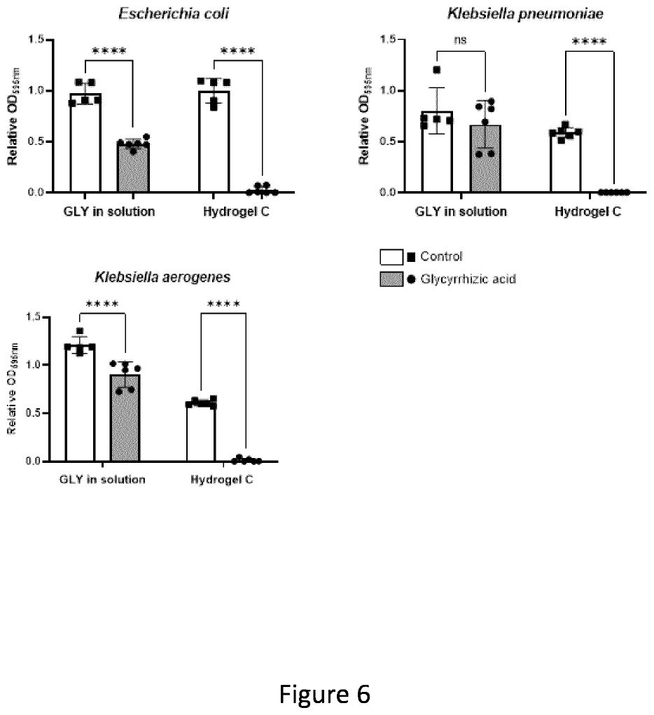

FIG. 7 Bactericidal activity of Hydrogel C

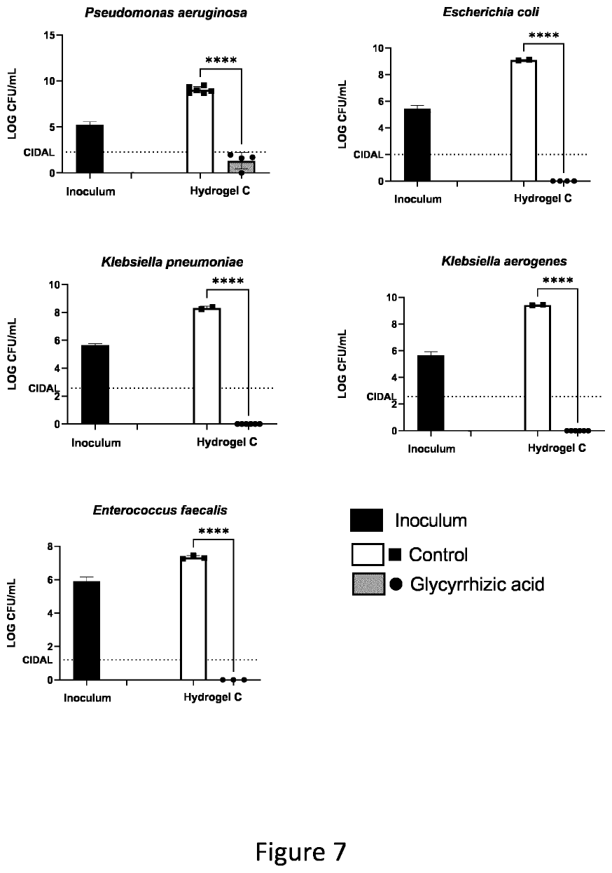

Abbreviations in the figures are: GLY: glycyrrhizic acid; Hydrogel A: a glycyrrhizic acid hydrogel with a gel pH of 4.2. This hydrogel acidifies the growth medium above the hydrogel (gel surface) to a pH of 5.2; Hydrogel B: a glycyrrhizic acid hydrogel with a gel pH of 4.2. This hydrogel acidifies the growth medium above the hydrogel (gel surface) to a pH of 4.8-5.0 depending on the percentage of glycyrrhizic acid in the hydrogel and the amount of this gel used in the assay; Hydrogel C: a glycyrrhizic acid hydrogel with a gel pH of 3.0. This hydrogel acidifies the growth medium above the hydrogel (gel surface) to a pH of 4.4-4.8 depending on the percentage of glycyrrhizic acid in the hydrogel and the amount of this gel used in the assay.

Hydrogel: According to the IUPAC gold Book, a hydrogel is defined as a gel in which the swelling agent is water with a finite, usually rather small yield stress. In the hydrogels of the present invention a network formed through physical interaction between molecules resulting in a thermo-reversible network as the regions of local order are thermally reversible. With respect to rheological parameters this means that the shear storage modulus (G′) needs to be higher than the loss modulus (G″). The G′ is preferentially 10 times higher than G″. Rheological measurements are performed on thermally stable hydrogels.

pH determination: The pH within the gel phase of a hydrogel in gel phase cannot be determined, and is therefore measured in the sol phase. Herein the gel itself becomes a liquid (G′≈G″) liquid by heating the gel above the degelation temperature and pH is measured at this temperature.

### Hydrogel

In a first aspect, the present invention relates to a glycyrrhizic acid hydrogel with a pH of between 2.5 and 5.0. The inventors have found that glycyrrhizic acid hydrogels in the specified pH range are more effective against bacterial infections. In particular, glycyrrhizic acid hydrogels at the specified pH range, unlike glycyrrhizic acid hydrogels at a different pH and unlike solutions containing glycyrrhizic acid, have shown to be effective against a broad spectrum of bacterial infections. In comparison, previous glycyrrhizic acid hydrogels and solutions containing glycyrrhizic acid were known to be effective against certain specific bacteria, while ineffective against many others.

In addition, the applicant found that the pH had a significant impact on the rheological properties of the hydrogel. Without being bound by theory, it is believed these rheological differences can be attributed to the extent to which glycyrrhizic acid is protonated. Glycyrrhizin contains three carboxyl groups, which can be protonated or deprotonated depending on the pH and this has an influence on e.g. the gel strength and the gelation temperature.

At a pH below 2.5, the glycyrrhizic acid hydrogel becomes brittle even at 37 °C, resulting in a gel that breaks or cracks easily. Furthermore, at lower pH glycyrrhizin and water must be mixed above the gelation range to obtain a homogeneous gel. As the gelation range increases in temperature with decreasing pH, this results in higher heating and energy requirements. These reasons made working at a pH below 2.5 increasingly impractical; as the gel became difficult to produce and apply.

As the pH increases, the gelation and degelation temperature decrease, gel strength and brittleness decrease. The sensitivity to changes and/or variations of the rheological properties to time, temperature, pH and glycyrrhizic acid concentration during gelation/degelation increase significantly.

At a pH above 5.0 gelation at room temperature required increasingly long periods of time. While gelation could be sped up by active cooling, this required more energy and equipment. Furthermore, minor deviations in temperature, pH or glycyrrhizic acid concentration had a large impact on the gel properties, including gelation times and gel strength. As a result, working at a pH above 5.0 quickly became increasingly impractical, as the gel became difficult to use and apply.

In the claimed ranges, water and glycyrrhizic acid can be mixed in the sol phase, that is to say above the degelation point, at relatively low temperatures. This is advantageous from an energetic and safety point of view. Upon cooling said sol phase, a stable and soft hydrogel is rapidly formed even at room temperature. This allows quick, safe and relatively easy production and use of the hydrogel. Present application achieves these features through modification of the pH rather than addition of additional gelators or cross-linking agents. This is particularly advantageous as it reduces issues with compatibility and in-vivo degradability of the glycyrrhizic acid hydrogel.

The term “glycyrrhizic acid hydrogel” as used herein refers to a hydrogel of glycyzzhizic acid, its derivatives, and combinations thereof. In a preferred embodiment, the glycyrrhizic acid hydrogel with a pH of between 2.5 and 5.0 comprises 2.5 to 25 wt. % of a molecule with formula (I) as depicted below or a pharmaceutically acceptable salt or solvate thereof:

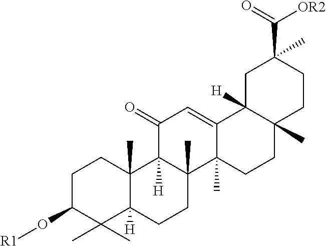

wherein R2 is H or a C1 to C20 alkyl chain;
and R1 is H or a glucuronic acid moiety depicted in formula (II)

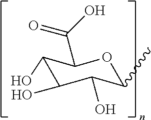

wherein n=1 to 10,
wherein the symbol [symbol] in formula (II) depicts the binding between the glucuronic acid comprising moiety and (I),
wherein if n=1, the glucuronic acid moiety is a glucuronic acid monomer,
wherein if n>1, the glucuronic acid comprising moiety is an alfa-1,2-linked glucuronic acid oligomer. In a preferred embodiment, R2 is a glucuronic acid moiety and n is 2.

In a preferred embodiment, R2 is H. In the most preferred embodiment, the molecule of formula (I) is glycyrrhizic acid.

In a preferred embodiment, the invention provides a glycyrrhizic acid hydrogel comprising 2.5 to 25 wt. % of glycyrrhizic acid, a pharmaceutically acceptable salt or solvate thereof, said hydrogel having a pH of between 2.5 and 5.0. In a further preferred embodiment, the glycyrrhizic acid hydrogel comprises 2.5 to 25 wt. % of a pharmaceutically acceptable salt of glycyrrhizic acid. Suitable pharmaceutically acceptable salts thereof may include alkali metal salts, e.g. sodium or potassium salts; alkaline earth metal salts, e.g. calcium or magnesium salts; and salts formed with suitable organic ligands, e.g. ammonium salts. Most preferably, the glycyrrhizic acid hydrogel comprises 2.5 to 25 wt. % of glycyrrhizic acid monoammonium salt.

In a preferred embodiment, the concentration of glycyrrhizic acid, a pharmaceutically acceptable salt or solvate thereof in said glycyrrhizic acid hydrogel is between 2 wt. % to 15 wt. %, more preferably between 2.5 and 15 wt. %, more preferably between 2.5 and 12 wt. %, more preferably between 2.5 and 10 wt. %, more preferably between 2.5 and 9 wt. %, more preferably between 3 and 8 wt. %, more preferably between 3 and 7 wt. %, more preferably between 4 and 7 wt. %, more preferably between 4 and 6 wt. %, most preferably about 5 wt. %. Advantageously, these concentrations of glycyrrhizic acid, a pharmaceutically acceptable salt or solvate thereof in water, at the pH range of 2 to 5 and in the absence of additional gelators results in a hydrogel with a gelation point between 40 and 60 °C. Advantageously compared to other hydrogels there is no need for an additional gelator, as glycyrrhizic acid acts as the gelator, thereby reducing toxicity and avoiding an immune response. Furthermore, it allows the hydrogel to be fully degradable and promotes independent API formulation by the physician.

In a preferred embodiment, the hydrogel is free of antibiotics. In a preferred embodiment, the hydrogel is free of antibacterials. In preferred embodiment, the hydrogel is free of anaesthetics. In a preferred embodiment, the hydrogel is free of active pharmaceutical ingredients. In the most preferred embodiment, the hydrogel is free of antibiotics, anaesthetics, antibacterials and active pharmaceutical ingredients. In other words, the hydrogel preferably does not contain any anbitiotics, antibacterials, anaesthetics or active pharmaceutical ingredients in addition to glycyrrhizic acid. In a further preferred embodiment, the hydrogel is administered in a formulation without antibiotics. In another further preferred embodiment, the hydrogel is administered in a formulation without further antibacterials. In another further preferred embodiment, the hydrogel is administered in a formulation without anaesthetics. In another further preferred embodiment, the hydrogel is administered in a formulation without further active pharmaceutical ingredients. In this context, free of anbitiotics, antibacterials, anaesthetics or active pharmaceutical ingredients refers to any anbitiotics, antibacterials, anaesthetics or active pharmaceutical ingredients in addition to glycyrrhizic acid. In a preferred embodiment, the hydrogel is free of antifungals. In another further preferred embodiment, the hydrogel is administered in a formulation without further antifungals.

This is advantageous as it limits the interactions between APIs; making it easier for medical professionals to determine treatment while remaining in full control of each API and its respective dose. Furthermore, many APIs influence the properties of the hydrogel. For example, tertiary amines commonly found in many APIs such as anaesthetics may increase gel hardness and the yield stress, which in turn impacts the efficacy of the hydrogel when treating bacterial infections.

The pH of the hydrogel can be modified to fall within the desired range using any suitable means. In a preferred embodiment, the pH is modified through the addition of pharmaceutically acceptable pH modifiers. In a preferred embodiment, the pH may be buffered in the desired range. This can be advantageous to ensure the pH remains within the desired range when the hydrogel is modified and/or applied. Preferably, pharmaceutically acceptable pH modifiers are chosen from pharmaceutically acceptable acids, pharmaceutically acceptable salts, pharmaceutically acceptable bases and pharmaceutically acceptable buffers. Pharmaceutically acceptable acids include hydrochloric acid, hydrobromic acid, sulphuric acid, phosphoric acid, nitric acid, acetic acid, maleic acid, fumaric acid, lactic acid, tartaric acid, citric acid and gluconic acid. Pharmaceutically acceptable salts include: hydrochloride, hydrobromide, sulphate, phosphate, nitrate, acetate, maleate, fumarate, lactate, tartrate, citrate and gluconate salts. Pharmaceutically acceptable bases include sodium hydroxide, potassium hydroxide, magnesium hydroxide, calcium hydroxide and aqueous ammonia NH₄OH. It is noted that these pH modifiers are used in a hydrogel and in order to obtain and/or maintain the pH in the desired range. In other words, these pH modifiers are heavily diluted and used in an aqueous medium. Pharmaceutically acceptable bases are suitable to adjust the pH as well as buffering the hydrogel at a desired pH; but it is noted that the desired pH remains well within the acidic range (i.e. lower than 5). The most preferred pH modifiers are chosen from the list of: HCl, HBr, NaOH and NH₄OH.

In a further preferred embodiment, the present invention provides a glycyrrhizic acid hydrogel essentially consisting of, preferably consisting of:
2.5 to 25 wt. % of glycyrrhizic acid, a pharmaceutically acceptable salt or solvate thereof;
pharmaceutically acceptable pH modifiers; in an amount so said hydrogel has a pH of between 2.5 and 5.0; and
water.

In another preferred embodiment, the present invention provides a glycyrrhizic acid hydrogel essentially consisting of, preferably consisting of:
2.5 to 25 wt. % of glycyrrhizic acid, a pharmaceutically acceptable salt or solvate thereof;
pharmaceutically acceptable pH modifiers chosen from: hydrochloric acid, hydrobromic acid, sulphuric acid, phosphoric acid, nitric, acetic acid, maleic acid, fumic acid, lactic acid, tartaric acid, citric acid, gluconic acid, hydrochloride salts, hydrobromide salts, sulphate salts, phosphate salts, nitrate salts, acetate salts, maleate salts, fumarate salts, lactate salts, tartrate salts, citrate salts gluconate salts, sodium hydroxide, potassium hydroxide, magnesium hydroxide, calcium hydroxide and aqueous ammonia NH₄OH; in an amount so said hydrogel has a pH of between 2.5 and 5.0; and
water.

In a preferred embodiment, the concentration of glycyrrhizic acid, a pharmaceutically acceptable salt or solvate thereof in said glycyrrhizic acid hydrogel is between 2 wt. % to 25 wt. %. More preferably, the concentration of glycyrrhizic acid, a pharmaceutically acceptable salt or solvate thereof is between 2.5 and 25 wt. %, more preferably the hydrogel comprises of 2.5%-3%; 2.5%-3.5%; 2.5%-4%; 2.5%-4.5%; 2.5%-5%; 2.5%-5.5%; 2.5%-6%; 2.5%-6.5%; 2.5%-7%; 2.5%-7.5%; 2.5%-8%; 2.5%-8.5%; 2.5%-9%; 2.5%-9.5%; 2.5%-10%; 2.5%-10.5%; 2.5%-11%; 2.5%-11.5%; 2.5%-12%; 2.5%-12.5%; 2.5%-13%; 2.5%-13.5%; 2.5%-14%; 2.5%-14.5%; 2.5%-15%; 2.5%-16%; 2.5%-17%; 2.5%-18%; 2.5%-19%; 2.5%-20%; 2.5%-21%; 2.5%-22%; 2.5%-23%; 2.5%-24%; 2.5%-25%; 3%-3.5%; 3%-4%; 3%-4.5%; 3%-5%; 3%-5.5%; 3%-6%; 3%-6.5%; 3%-7%; 3%-7.5%; 3%-8%; 3%-8.5%; 3%-9%; 3%-9.5%; 3%-10%; 3%-10.5%; 3%-11%; 3%-11.5%; 3%-12%; 3%-12.5%; 3%-13%; 3%-13.5%; 3%-14%; 3%-14.5%; 3%-15%; 3%-16%; 3%-17%; 3%-18%; 3%-19%; 3%-20%; 3%-21%; 3%-22%; 3%-23%; 3%-24%; 3%-25%; 3.5%-4%; 3.5%-4.5%; 3.5%-5%; 3.5%-5.5%; 3.5%-6%; 3.5%-6.5%; 3.5%-7%; 3.5%-7.5%; 3.5%-8%; 3.5%-8.5%; 3.5%-9%; 3.5%-9.5%; 3.5%-10%; 3.5%-10.5%; 3.5%-11%; 3.5%-11.5%; 3.5%-12%; 3.5%-12.5%; 3.5%-13%; 3.5%-13.5%; 3.5%-14%; 3.5%-14.5%; 3.5%-15%; 3.5%-16%; 3.5%-17%; 3.5%-18%; 3.5%-19%; 3.5%-20%; 3.5%-21%; 3.5%-22%; 3.5%-23%; 3.5%-24%; 3.5%-25%; 4%-4.5%; 4%-5%; 4%-5.5%; 4%-6%; 4%-6.5%; 4%-7%; 4%-7.5%; 4%-8%; 4%-8.5%; 4%-9%; 4%-9.5%; 4%-10%; 4%-10.5%; 4%-11%; 4%-11.5%; 4%-12%; 4%-12.5%; 4%-13%; 4%-13.5%; 4%-14%; 4%-14.5%; 4%-15%; 4%-16%; 4%-17%; 4%-18%; 4%-19%; 4%-20%; 4%-21%; 4%-22%; 4%-23%; 4%-24%; 4%-25%; 4.5%-5%; 4.5%-5.5%; 4.5%-6%; 4.5%-6.5%; 4.5%-7%; 4.5%-7.5%; 4.5%-8%; 4.5%-8.5%; 4.5%-9%; 4.5%-9.5%; 4.5%-10%; 4.5%-10.5%; 4.5%-11%; 4.5%-11.5%; 4.5%-12%; 4.5%-12.5%; 4.5%-13%; 4.5%-13.5%; 4.5%-14%; 4.5%-14.5%; 4.5%-15%; 4.5%-16%; 4.5%-17%; 4.5%-18%; 4.5%-19%; 4.5%-20%; 4.5%-21%; 4.5%-22%; 4.5%-23%; 4.5%-24%; 4.5%-25%; 5%-5.5%; 5%-6%; 5%-6.5%; 5%-7%; 5%-7.5%; 5%-8%; 5%-8.5%; 5%-9%; 5%-9.5%; 5%-10%; 5%-10.5%; 5%-11%; 5%-11.5%; 5%-12%; 5%-12.5%; 5%-13%; 5%-13.5%; 5%-14%; 5%-14.5%; 5%-15%; 5%-16%; 5%-17%; 5%-18%; 5%-19%; 5%-20%; 5%-21%; 5%-22%; 5%-23%; 5%-24%; 5%-25%; 6%-7%; 6%-8%; 6%-9%; 6%-10%; 6%-11%; 6%-12%; 6%-13%; 6%-14%; 6%-15%; 6%-16%; 6%-17%; 6%-18%; 6%-19%; 6%-20%; 6%-21%; 6%-22%; 6%-23%; 6%-24%; 6%-25%; 7%-8%; 7%-9%; 7%-10%; 7%-11%; 7%-12%; 7%-13%; 7%-14%; 7%-15%; 7%-16%; 7%-17%; 7%-18%; 7%-19%; 7%-20%; 7%-21%; 7%-22%; 7%-23%; 7%-24%; 7%-25%; 8%-9%; 8%-10%; 8%-11%; 8%-12%; 8%-13%; 8%-14%; 8%-15%; 8%-16%; 8%-17%; 8%-18%; 8%-19%; 8%-20%; 8%-21%; 8%-22%; 8%-23%; 8%-24%; 8%-25%; 9%-10%; 9%-11%; 9%-12%; 9%-13%; 9%-14%; 9%-15%; 9%-16%; 9%-17%; 9%-18%; 9%-19%; 9%-20%; 9%-21%; 9%-22%; 9%-23%; 9%-24%; 9%-25%; 10%-12%; 10%-14%; 10%-16%; 10%-18%; 10%-20%; 10%-22%; 10%-25%; 12%-15%; 12%-18%; 12%-21%; 12%-25%; 14%-18%; 14%-21%; 14%-23%; 14%-25%; 16%-19%; 16%-21%; 16%-23%; 16%-25%; or 25% glycyrrhizic acid, a pharmaceutically acceptable salt or solvate thereof in water. The wording “% of a molecule” as described herein refers to wt. % or percentage by weight.

In a preferred embodiment, the glycyrrhizic acid hydrogel has a temperature lower than 40 °C. That is to say, the glycyrrhizic acid hydrogel is a stable hydrogel at temperatures lower than 40 °C. In a preferred embodiment, the glycyrrhizic acid hydrogel has a gelation point higher than 40 °C. In a preferred embodiment, the glycyrrhizic acid hydrogel has a degelation point higher than 40 °C. It is strongly preferred that the degelation point of the hydrogel is higher than the body temperature of the mammal to be treated for a wound otherwise the hydrogel will become liquid when applied onto the wound. Present application shows that the hydrogel form has significant beneficial effects for use in treatment or prevention of bacterial infections than a liquid form. For example the gelation point for the 2.5% Glycyrrhizic acid hydrogel was determined to be 40 °C in the experimental setup of the examples of the present invention, while the gelation point for the 5% glycyrrhizic acid hydrogel was determined to be at 45 °C, under these experimental conditions.

The “gelation point” or “gelation temperature” as used herein is defined as the temperature at which the elastic modulus G′ becomes larger than the loss modulus G″, measured while cooling down at a rate of 0.01 °C/s. The “degelation point” or “degelation temperature” is defined as the temperature at which the loss modulus G″ becomes larger than the elastic modulus G′, measured while heating up at a rate of 0.01 °C/s.

Glycyrrhizic acid hydrogels gelation and degelation show hysteresis, so the gelation and degelation point and degelation point are not a single value. The “gelation range” or “degelation range” as used herein is the interval defined by the gelation point and the degelation point; both measured with a temperature sweep of 0.01 °C/s.

Preferably, the gelation and degelation range lies entirely above the body temperature of the mammal to be treated, more preferably above 40 °C. In a further preferred embodiment, the degelation range lies entirely between 40 and 70 °C, more preferably the degelation range lies entirely between 40 and 60 °C, more preferably the degelation range lies entirely between 40 and 50 °C. The minimum temperature of 40 °C is beneficial to ensure the hydrogel remains a hydrogel, which is highly desirable for its antibacterial and wound-dressing functions. The maximum temperature is beneficial to allow easy, safe and less energy-intensive processability of the hydrogel in its sol phase.

### Treatment

In a second aspect, the present invention provides a glycyrrhizic acid hydrogel as broad-spectrum hydrogel composition for use in the treatment or prevention of infections, preferably skin and soft tissue infections. In a preferred embodiment, the present invention provides a glycyrrhizic acid hydrogel with a pH below 5.0, for use in the treatment or prevention of an infection, preferably bacterial infection. In a more preferred embodiment, the invention provides a glycyrrhizic acid hydrogel comprising 2.5 to 25 wt. % of glycyrrhizic acid, a pharmaceutically acceptable salt or solvate thereof, said hydrogel having a pH of between 2.5 and 5.0, for use in the treatment or prevention of a bacterial infection. In a preferred embodiment, the invention relates to a hydrogel composition for use in the treatment or prevention of infections in humans or animals, most preferably in humans.

In a further aspect, the present invention provides a method of treating or preventing a bacterial infection in an individual comprising the step of administering an effective amount of the glycyrrhizic acid hydrogel of the first or second aspect. In a preferred embodiment, the present invention provides a method of treating or preventing a bacterial infection in an individual comprising the step of administering an effective amount of the glycyrrhizic acid hydrogel, a pharmaceutically acceptable salt or solvate thereof, with a pH of between 2.5 and 5.0.

The applicant has surprisingly found that acidifying a glycyrrhizic acid hydrogel increases the activity spectrum of said gel against bacterial infection, as well as modifying the rheological properties of the gel.

In a preferred embodiment, the glycyrrhizic acid hydrogel has a pH lower than 5.0, more preferably a pH lower than 4.9, more preferably a pH lower than 4.8, more preferably a pH lower than 4.85, more preferably a pH lower than 4.8, more preferably a pH lower than 4.75, more preferably a pH lower than 4.7, more preferably a pH lower than 4.6, more preferably a pH lower than 4.5, more preferably a pH lower than 4.4, more preferably a pH lower than 4.3, more preferably a pH lower than 4.2, more preferably a pH lower than 4.0, more preferably a pH lower than 3.9, more preferably a pH lower than 3.8, more preferably a pH lower than 3.7, more preferably a pH lower than 3.6, more preferably a pH lower than 3.5, more preferably a pH lower than 3.4, more preferably a pH lower than 3.3, more preferably a pH lower than 3.2.

Preferably, the glycyrrhizic acid hydrogel has a pH of at least 1.0, more preferably a pH of at least 2.0, more preferably a pH of at least 2.1, more preferably a pH of at least 2.2, more preferably a pH of at least 2.3, more preferably a pH of at least 2.4, more preferably a pH of at least 2.5, more preferably a pH of at least 2.6, more preferably a pH of at least 2.7, more preferably a pH of at least 2.8, more preferably a pH of at least 2.9, more preferably a pH of at least 3.0, more preferably a pH of at least 3.1, more preferably a pH of at least 3.2.

In a preferred embodiment, the glycyrrhizic acid hydrogel has a pH between 1.0 and 5.0, more preferably a pH between 2.0 and 5.0, more preferably a pH between 2.5 and 5.0, more preferably a pH between 2.6 and 4.9, more preferably a pH between 2.7 and 4.8, more preferably a pH between 2.8 and 4.8, more preferably a pH between 2.8 and 4.5, more preferably a pH between 2.8 and 4.2, more preferably a pH between 2.8 and 4.0, more preferably a pH between 2.8 and 3.9.

The applicant surprisingly found that the claimed pH window increases the efficacy of a glycyrrhizic acid hydrogel against bacterial infections. Furthermore, rather than being effective only against particular bacterial infections outside of the preferred pH range, a broad spectrum of antibacterial activity was achieved. This is advantageous as it allows the use of glycyrrhizic acid hydrogel against a wider range of bacterial infections, as well as allowing the use of glycyrrhizic acid hydrogel as a first treatment without or before testing which bacteria are causing the infection.

In a further preferred embodiment, the glycyrrhizic acid hydrogel has a pH between 4.0 and 4.4, more preferably a pH between 4.1 and 4.3, most preferably a pH of about 4.2. In a further preference, this hydrogel is for use in the treatment or prevention of a bacterial invention, wherein the bacterial infection is of MRSA, Staphylococcus epidermidis, Acinetobacter baumanni, E. faecalis or S. pyogenes. More preferably, the bacterial infection is of MRSA, Acinetobacter baumannii and Streptococcus pyogenes. This particular pH range was found to be the most effective against a bacterial infection of MRSA, Acinetobacter baumannii and Streptococcus pyogenes. Furthermore, this pH range was found very effective against bacterial infection of MRSA, Acinetobacter baumannii and Streptococcus pyogenes.

In a further preferred embodiment, the glycyrrhizic acid hydrogel has a pH between 3.6 and 4.0, more preferably a pH between 3.7 and 3.7, most preferably a pH of about 3.8. In a further preference, this hydrogel is for use in the treatment or prevention of a bacterial invention, wherein the bacterial infection is of Pseudomonas aeruginosa. This particular pH range was found to be the most effective against a bacterial infection of Pseudomonas aeruginosa.

In another preferred embodiment, the glycyrrhizic acid hydrogel has a pH between 2.8 and 3.2, more preferably a pH between 2.9 and 3.2, most preferably a pH of about 3. In a further preference, this hydrogel is for use in the treatment or prevention of a bacterial infection, wherein the bacterial infection is of Pseudomonas aeruginosa, Escherichia coli, Klebsiella pneumoniae, Klebsiella aerogenes or Enterococcus faecalis. Most preferably, this hydrogel is for use in the treatment or prevention of a bacterial infection wherein the bacterial infection is of Escherichia coli, Klebsiella pneumoniae or Klebsiella aerogenes. This particular pH range was found to be the most effective against a bacterial infection of found to be the most effective against a bacterial infection of Escherichia coli, Klebsiella pneumoniae or Klebsiella aerogenes. Furthermore, this pH range was found very effective against bacterial infection of Pseudomonas aeruginosa, Escherichia coli, Klebsiella pneumoniae, Klebsiella aerogenes or Enterococcus faecalis.

In a preferred embodiment, the hydrogel is for topical use. In a further preferred embodiment, the hydrogel is for use in the treatment or prevention of a bacterial infection, wherein the hydrogel is for topical use. In a further preferred embodiment, the hydrogel is for topical use on chronic wounds. The term “chronic wound” refers to a wound that has not healed. Wounds that do not heal within 6 weeks, for example, are considered chronic. Chronic wounds include, for example, pressure ulcers, decubitus ulcers, diabetic ulcers including diabetic foot and leg ulcers, slow or non-healing venous ulcers, venous stasis ulcers, arterial ulcers, vasculitic ulcers, burn ulcers, trauma-induced ulcers, infectious ulcers, mixed ulcers, and pyoderma gangrenosum. The chronic wound may be an arterial ulcer that comprises ulcerations resulting from complete or partial arterial blockage. The chronic wound may be a venous or venous stasis ulcer that comprises ulcerations resulting from a malfunction of the venous valve and the associated vascular disease. Bacterial infections commonly occur in chronic wounds as they provide suitable entry and growth locations for bacteria. The glycyrrhizic acid hydrogel of present invention advantageously may be used to treat or prevent bacterial infections over a broad spectrum of bacteria.

In another preferred embodiment, the hydrogel is for internal use. In a further preferred embodiment, the hydrogel is for use in the treatment or prevention of bacterial infection, wherein the hydrogel is for internal use.

In another preferred embodiment, the hydrogel is applied as a coating on a medical device. In a further preferred embodiment, the hydrogel is for use in the treatment or prevention of a bacterial infection wherein the hydrogel is applied as a coating on a medical device.

A non-limited list of conditions and bacteria to be treated with the hydrogels of the present invention are listed in table 1.

TABLE 1
Examples of etiological risk factors for skin and soft
tissue infections and their associated bacterial causes

| Risk factor | Associated etiological pathogen |
|---|---|
| Diabetes | Staphylococci, group B streptococci, Acinetobacter |
| mellitus | baumannii, Eschericia coli |
| Cirrhosis | Klebsiella pneumoniae, Klebsiella aerogenes, |
| | Escherichia coli, Acinetobacter baumannii |
| Neutropenia | Pseudomonas aeruginosa |
| Hot tub | Pseudomonas aeruginosa |
| exposure/ | |
| loofah sponge | |
| IV drug abuse | Methicillin-resistant Staphylococcus aureus (MRSA), |
| | Pseudomonas aeruginosa |

### EXAMPLES

### Example 1: Decreasing the pH of the Glycyrrhizic Acid Hydrogel Increases the Activity Spectrum

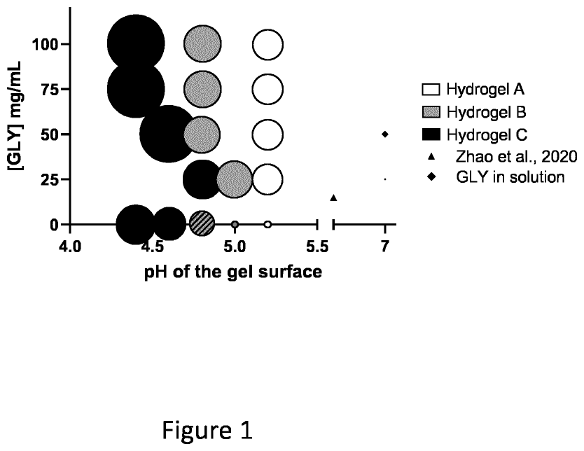

The circles in FIG. 1 represent the number of pathogens (9 tested in total) of which the growth is completely inhibited by the specific hydrogel (defined according to glycyrrhizic acid concentration and gel surface pH). The circle size is proportional to the number of pathogens of which the growth is completely inhibited. The glycyrrhizic acid concentration is calculated based on the percentage of glycyrrhizic acid in the gel and the amount of this gel used in the assay. Note that the pH at the gel surface is lower than the initial pH of the growth medium on top of the gel due to the acidifying effect of the hydrogel. This pH difference is dependent of the pH of the hydrogel, the percentage of glycyrrhizic acid in the hydrogel and the amount of hydrogel used. The different hydrogels are indicated by color: white circles represent the hydrogel A, grey circles represent hydrogel B and black circles represent the hydrogel C. The 2 grey dots at pH 7.0 in the plot represent the (lack of) activity of glycyrrhizic acid in solution. Of all nine pathogens tested, only Streptococcus pyogenes was inhibited by 50 mg/mL glycyrrhizic acid in solution.

The black triangle at pH 6.5 in the plot represent the activity of the hydrogel prepared as described in (Zhao et al. (2020) cited above). Herein the hydrogel of Zhao et al was prepared according to the instructions of the authors and pH measurement was done as detailed in the methods and materials of the present invention.

### Example 2: Decreasing the pH of the Glycyrrhizic Acid Hydrogel Increases the Antibacterial Activity

The below table summarises the antibacterial effect (no effect, bacteriostatic, bactericidal) of the glycyrrhizic acid hydrogels on different pathogens tested.

TABLE 2
Overview of the antibacterial effect (no effect,
bacteriostatic, bactericidal) of the glycyrrhizic
acid hydrogels on different pathogens tested.

| | GLY in | Hydrogel | Hydrogel | |
|---|---|---|---|---|
| | solution | A | B | Hydrogel C |
| MRSA | No effect | Bactericidal | Bactericidal | Bactericidal |
| S. epidermidis | No effect | Bacterio- | Bactericidal | Bactericidal |
| | | static | | |
| A. baumannii | No effect | Bactericidal | Bactericidal | Bactericidal |
| P. aeruginosa | No effect | No effect | Bacterio- | Bactericidal |
| | | | static | |
| E. coli | No effect | No effect | No effect | Bactericidal |
| | | | | (>50 mg/mL) |
| K. pneumoniae | No effect | No effect | No effect | Bactericidal |
| | | | | (>50 mg/mL) |
| K. aerogenes | No effect | No effect | No effect | Bactericidal |
| | | | | (>50 mg/mL) |
| Streptococcus | Bacterio- | Bactericidal | Bactericidal | Bactericidal |
| | static | | | |
| Enterococcus | No effect | Bacterio- | Bacterio- | Bactericidal |
| | | static | static | |

### Example 3: Growth Inhibiting Activity of Hydrogel A

Hydrogel A shows increased growth inhibiting activity against MRSA, Staphylococcus epidermidis, Acinetobacter baumanni, E. faecalis and S. pyogenes, as compared to the lack of/limited growth inhibiting activity of glycyrrhizic acid in solution or at the corresponding pH associated with the acidification of the environment caused by glycyrrhizic acid hydrogels.

The growth inhibiting effect of 25 mg/mL glycyrrhizic acid in solution and 0.5 mL/mL of 5 wt % hydrogel A, corresponding with a total of 25 mg/mL of glycyrrhizic acid in the system are shown in grey bars with circles. Both are compared to suitable controls (shown in white bars with squares) i.e. growth media with an adjusted pH that is associated with the acidifying effect of glycyrrhizic acid in solution (pH 7.4) and hydrogel A (pH 5.2) but without the presence of glycyrrhizic acid in solution or hydrogel A, respectively. ns: p>0.05, *p<0.05, **p<0.01, ****p<0.0001.

### Example 4: Bactericidal Activity of Hydrogel A

Hydrogel A shows bactericidal activity against MRSA, Acinetobacter baumannii and Streptococcus pyogenes.

Bactericidal activity is defined as a 3 LOG reduction compared to the inoculum (black bars). The bactericidal effect of 0.5 mL/mL of 5 wt % hydrogel A (shown in grey bars with circles) is compared to growth medium with an adjusted pH (pH 5.2) that is associated with the acidifying effect of hydrogel A (shown in white bars with squares). ns: p>0.05, ****p<0.0001.

### Example 5: Growth Inhibiting Activity of Hydrogel B

Hydrogel B shows increased growth inhibiting activity against Pseudomonas aeruginosa, as compared to the lack of/limited growth inhibiting activity of glycyrrhizic acid in solution or at the corresponding pH associated with the acidification of the environment caused by glycyrrhizic acid hydrogels.

The growth inhibiting effect of 25 mg/mL glycyrrhizic acid in solution and 0.5 mL/mL of 5 wt % hydrogel B, corresponding with a total of 25 mg/mL of glycyrrhizic acid in the system are shown in grey bars with circles. Both are compared to suitable controls (shown in white bars with squares) i.e. growth media with an adjusted pH that is associated with the acidifying effect of glycyrrhizic acid in solution (pH 7.4) and hydrogel B (pH 5) but without the presence of glycyrrhizic acid in solution or hydrogel B, respectively. ns: p>0.05, ****p<0.0001.

### Example 6: Bactericidal Activity of Hydrogel B Shows

Hydrogel B shows bactericidal activity against Staphylococcus epidermidis. Bactericidal activity is defined as a 3 LOG reduction compared to the inoculum (black bars). The bactericidal effect of 0.5 mL/mL of 5 wt % hydrogel B (shown in grey bars with circles) is compared to growth medium with an adjusted pH (pH 5) that is associated with the acidifying effect of hydrogel B (shown in white bars with squares). ****p<0.0001.

### Example 7: Growth Inhibiting Activity of Hydrogel C

Hydrogel C shows increased growth inhibiting activity against Escherichia coli, Klebsiella pneumoniae and Klebsiella aerogenes, as compared to the lack of/limited growth inhibiting activity of glycyrrhizic acid in solution or at the corresponding pH associated with the acidification of the environment caused by glycyrrhizic acid hydrogels.

The growth inhibiting effect of 50 mg/mL glycyrrhizic acid in solution and 0.5 mL/mL of 10 wt % hydrogel C, corresponding with a total of 50 mg/mL of glycyrrhizic acid in the system are shown in grey bars with circles. Both are compared to suitable controls (shown in white bars with squares) i.e. growth media with an adjusted pH that is associated with the acidifying effect of glycyrrhizic acid in solution (pH 7.4) and hydrogel C (pH 4.6) but without the presence of glycyrrhizic acid in solution or hydrogel C, respectively. ns: p>0.05, ****p<0.0001.

### Example 8: Bactericidal Activity of Hydrogel C

Hydrogel C shows bactericidal activity against Pseudomonas aeruginosa, Escherichia coli, Klebsiella pneumoniae, Klebsiella aerogenes and Enterococcus faecalis. Bactericidal activity is defined as a 3 LOG reduction compared to the inoculum (black bars). The bactericidal effect of 0.5 mL/mL of 10 wt % hydrogel C (shown in grey bars with circles) is compared to growth medium with an adjusted pH (pH 4.6) that is associated with the acidifying effect of hydrogel C (shown in white bars with squares). ****p<0.0001.

### Examples 9: Materials and Methods

### Strains and Chemicals

Escherichia coli UTI89, Pseudomonas aeruginosa LMG9009, Staphylococcus aureus ATCC6538, Klebsiella pneumoniae ATCC13883, Klebsiella aerogenes ATCC13048, Acinetobacter baumannii RD5SR3, Enterococcus faecalis LMG8148, Streptococcus pyogenes LMG 14237 and clinical isolates of a Staphylococcus epidermidis and a methicillin resistant S. aureus (MRSA), isolated from osteomyelitis patients were used. Prior to the experiments, all strains, except S. pyogenes were inoculated in Lysogeny broth (LB) and incubated overnight on 37 °C (200 rpm). Overnight cultures of S. pyogenes were grown micro-aerophilic in Brain-Heart infusion broth (BHI). Thereafter, the bacteria were normalized to roughly 5*10⁵ CFU mL⁻¹.

Lysogeny broth (LB; 10 g L⁻¹ NaCl, 10 g L⁻¹ Tryptone, 5 g L⁻¹ Yeast Extract) was used to create overnight cultures (ONC). Mueller Hinton Broth (MHB; 21 g L⁻¹ MHB) was used for the minimal inhibitory concentration (MIC) assays for all strain except S. pyogenes. For this strain, BHI was used. If LB agar, BHI agar or MHB agar was needed, 15 g L⁻¹ of bacteriological agar was added. MHB or BHI batches with varying pH were made by adding HCl or NaOH to acidify or alkalify the medium to the desired pH-value, respectively.

Three different formulations of the glycyrrhizic acid hydrogels were made in dH₂O: (i) Hydrogel C with pH=3, (ii) Hydrogel B with pH=3.8 and (iii) Hydrogel A with pH=4.2. pH was measured at 70 °C and varied using HCl or NaOH to acidify or alkalify the formulation to the desired pH-value. Moreover, different concentration batches (mass by volume) were used for the hydrogels: 2.5%, 5%, 7.5%, 10%, 15% and 20%. Glycyrrhizic acid in solution—not a hydrogel—was prepared at pH 7.4.

### Growth Inhibition Evaluation

To determine the growth inhibiting activity of glycyrrhizic acid in solution, 100 μL of different 1:2 dilutions of a 100 mg/mL stock solution of glycyrrhizic acid in MHB were added to the wells of a 96 well microtiter plate, after which 100 μL of a 5*10⁵ CFU mL⁻¹ bacteria suspension in MHB was added to each well.

To determine the growth inhibiting activity of the glycyrrhizic acid hydrogels, the hydrogel was liquefied by heating to 70 °C. When liquid, varying volumes of the liquid hydrogel (80 μL to 160 μL) were pipetted into a 96 well microtiter plate. After the hydrogel solidified, 120 μL of a 5*10⁵ CFU mL⁻¹ bacteria suspension in MHB was added on top of the hydrogel.

To avoid evaporation, the plates were sealed using a membrane (Greiner Bio-one NV). After 24 hours of incubation (37 °C, 200 rpm), the OD595 was measured and relative OD595 was calculated by dividing the OD595 by the average OD595 of the growth control (=growth of cells in MHB without glycyrrhizic acid).

### Biocidal Activity Evaluation

To determine the biocidal activity of glycyrrhizic acid in solution and in hydrogel, at the end of the growth inhibition assay (see above), after incubation, 100 μL of the medium on top of the hydrogel was diluted 10 times in Phosphate saline buffer (PBS; 1.24 g L⁻¹ K₂HPO₄, 0.39 g L⁻¹ KH₂PO₄, 8.8 g L⁻¹ NaCl) and plated out on MHB agar plates by spreading 100 μL of the dilution. After incubating overnight at 37 °C, colonies were counted and the colony forming units were determined.

### Rheological Evaluation

The rheological properties of glycyrrhizic acid hydrogels over the pH range of 2 to 5 were evaluated during time, strain, frequency and temperature sweeps on a stress controlled rheometer. Additionally, DSC measurements were performed.

It was found that the strength of the hydrogels decreases with increasing pH. At pH 3.00 and lower, microcracks start forming inside the material. This resulted in a hard but increasingly brittle hydrogel. The differences in rheological properties between 20 °C and 37 °C at pH 3 were small. Gelation starts almost instantly, and the moduli reach steady-state in less than 5 minutes.

Furthermore, mixing of glycyrrhizic acid and water quickly forms an impermeable gel; making it impossible for the mixture to gelate completely. To obtain a homogeneous gel, the mixture should be heated to above its degelation temperature and adequately mixed in the sol phase. As the gelation temperature and degelation temperature both increase with decreasing pH; lower pH results in higher heating requirements. These reasons made working with a hydrogel with pH below 2.5 impractical.

As the pH increases, the hydrogels become softer and less brittle. Furthermore, it takes longer for the samples to gelate. In other words, it takes longer for the moduli to reach steady-state with increasing pH. The gelation temperature decreases with increasing pH. At a pH above 5.35 and a glycyrrhizin concentration of 10 wt. %, the gelation temperature is under room temperature. The gels are relatively frequency independent at every pH.

At pH near 5 and above, the effects of temperature and time on gel formation become increasingly large. For example, at a (monoammonium) glycyrrhizin concentration of 10 wt. % in water, adjusted in pH with HCl and NH₄OH, there is an enormous difference in moduli and gelation time between pH 4.70 and pH 4.85 at 20 °C. While both pH result in a soft hydrogel in steady state; reaching G′ of 100 Pa takes 5 minutes 30 seconds at a pH of 4.70. Reaching the same G′ of 100 Pa takes 75 minutes at pH 4.85.

Due to the drastically increasing gelation times as well as the sensitivity of the gel to differences in pH, temperature and glycyrrhizic acid concentration; using glycyrrhizic acid hydrogels at a pH above 5 becomes increasingly impractical. Gelation may be sped up through cooling, but this increases the necessary equipment and energy requirements for preparation. Furthermore, this was found to exacerbates issues with a high temperature sensitivity when the hydrogel is applied to a wound. The rapid substantial decrease in gel strength due to the increased temperature resulted in difficult handling.

1.-17. (canceled)

18. A method of treating or preventing a bacterial infection in an individual comprising administering a hydrogel that includes an effective amount of glycyrrhizic acid, a pharmaceutically acceptable salt or solvate thereof, with a pH of between 2.5 and 5.0.

19. The method according to claim 18, wherein the hydrogel has a pH between 2.8 and 4.2.

20. The method according to claim 18, wherein the hydrogel has a pH between 4.0 and 4.4, or has a pH between 4.1 and 4.3, or has a pH of 4.2.

21. The method according to claim 19, wherein the bacterial infection is of MRSA, Staphylococcus epidermidis, Acinetobacter baumanni, E. faecalis or S. pyogenes.

22. The method according to claim 19, wherein the bacterial infection is of MRSA, Acinetobacter baumannii and Streptococcus pyogenes.

23. The method according to claim 18, wherein the hydrogel has a pH between 3.6 and 4.0, or has a pH between 3.7 and 3.9, or has a pH of 3.8.

24. The method according to claim 22, wherein the bacterial infection is of Pseudomonas aeruginosa.

25. The method according to claim 18, wherein the hydrogel has a pH between 2.8 and 3.2 or has a pH between 2.9 and 3.1 or has a pH of 3.

26. The method according to claim 24, wherein the bacterial infection is of Pseudomonas aeruginosa, Escherichia coli, Klebsiella pneumoniae, Klebsiella aerogenes or Enterococcus faecalis.

27. The method according to claim 24, wherein the bacterial infection is of Escherichia coli, Klebsiella pneumoniae or Klebsiella aerogenes.

28. The method according to claim 18, wherein the hydrogel is administered topically.

29. The method according to claim 18, wherein the hydrogel is administered internally.

30. The method according to claim 18, wherein the administering includes using the hydrogel applied as a coating on a medical device.

31. The method according to claim 18, wherein the hydrogel is administered in a formulation without antibacterial agents.

32. The method according to claim 18, wherein a concentration of the glycyrrhizic acid, the pharmaceutically acceptable salt or solvate thereof, in the hydrogel is between 2 wt. % to 25 wt. %.

33. The method according to claim 18, wherein the hydrogel is administered in a formulation without antifungal agents.
